(19) United States
(12) Patent Application Publication
(10) Pub. No.: US 2005/0084861 A1
(43) Pub. Date: Apr. 21, 2005

(54) Methods of detecting modification of genetic material and monitoring processes thereof

(76) Inventors: Ian Weeks, Cardiff (GB); Richard Charles Brown, Cardiff (GB); Andrew Morby, Cardiff (GB); Colin Berry, Cardiff (GB)

Correspondence Address: GEN PROBE INCORPORATED, 10210 GENETIC CENTER DRIVE, SAN DIEGO, CA, 92121, US

(21) Appl. No.: 10/499,223

(22) PCT Filed: Dec. 17, 2002
(86) PCT No.: PCT/GB02/05724

(30) Foreign Application Priority Data
Dec. 19, 2001 (GB) 0130268.6

(51) Int. Cl. C12Q 1/68

(52) U.S. Cl. 435/6

(57) ABSTRACT

The invention relates to a method for determining the activity of an enzyme or enediyne capable of altering the structure of a “substrate” nucleic acid from a first to a second state wherein the activity of the enzyme or enedyine is monitored using a chemiluminescent label that is either attached to the “substrate” nucleic acid or an oligonucleotide which is complementary thereto or the enzyme or enediyne product thereof.

### FIELD OF THE INVENTION

This invention relates to a method of detecting and/or quantifying the activity of enzymes involved in the modification of genetic material. The method is based on the use of labelled nucleic acids, wherein the labels used may be, for example, fluorescent or chemiluminescent molecules and the chemical properties of said labels may be modified depending upon the state of the nucleic acid in which the label is situated, either ab initio or as a result of a hybridisation step. The invention also extends to the use of the method in screening for pharmacological agents; agents identified thereby; and synthetic nucleic acid enzyme substrates.

### BACKGROUND OF THE INVENTION

The replication, recombination, repair and other modification of deoxyribonucleic acid (DNA) and ribonucleic acid (RNA) molecules all involve changes in the structure of genetic material and are of fundamental importance to all living organisms. Examples of such processes are enzymatic reactions where the enzymes are ligase, nuclease, integrase, transposase, helicase, gyrase, polymerase, primase, reverse transcriptase and. For example, DNA ligases are enzymes involved in the modification of nucleic acid in organisms and can be divided into two classes, (i) the eukaryotic and viral enzymes which are ATP dependent, and (ii) the prokaryotic DNA ligases, which are dependent on NADH. In addition, prokaryotic ligases are unable to ligate blunt ended fragments and these distinct features of the prokaryotic enzymes make them an attractive target for selective antibiosis. Work on eukaryotic systems has also indicated that lack of ligase activity in humans correlates with certain pathological conditions.

The importance of assessing the activity of these enzymes has therefore led to attempts to develop assay systems for the detection of factors affecting nucleic acid. Of particular interest is the ability to monitor bacterial or viral enzyme activity in the screening of novel compounds either singly or in combination for anti-bacterial or anti-viral properties due to their ability to inhibit the enzyme.

### DESCRIPTION OF THE PRIOR ART

Current assays for assessing the activity of these enzymes, for example ligase (FIG. 1 of the accompanying drawings) involve measurement of enzyme intermediates or structural characterisation of the substrate and/or product. The majority of these assays also employ radioactivity, necessitating additional experimental precautions and incurring costs for waste disposal. Recent assays have attempted to replace the use of radioactivity with fluorescent labels for enzyme substrates. Alternatively, biological assays for DNA ligase activity have been developed but are time consuming (at least 2 days), laborious and qualitative rather than quantitative. A more rapid biological assay has been described (U.S. Pat. No. 5,976,806) but this involves the use of coupled transcription-translation systems with expression of a reporter gene product (e.g. luciferase) in addition to the DNA ligase, making this multi-enzyme/multi-stage assay unsuitable for the high-throughput screening of potential pharmaceutical compounds.

Assays for helicase (FIG. 2 of the accompanying drawings) have been described which exploit the unwinding of double stranded nucleic acid. In one example (U.S. Pat. No. 5,958,696), a solid-phase derivative of a double-stranded nucleic acid is prepared in which one of the strands is labelled with a radioisotope. Helicase activity is detected by its ability to release the labelled strand into the solution phase which can be separated and measured. In a further example use is made of the ability of certain markers to associate preferentially with multi-stranded, e.g. double stranded nucleic acid, as opposed to single stranded nucleic acid. Thus the marker will not associate with material which is unwound by helicase activity.

The use of direct chemiluminescent-labelled oligonucleotide probes has been described for the quantification of RNA in infectious organisms (U.S. Pat. No. 5,283,174, U.S. Pat. No. 5,399,491). In particular, these methods have shown utility in the detection of target nucleic acid sequences since use is made of the fact that certain molecules are protected from degradation when associated with a nucleic acid duplex such that they retain an identifiable property as compared with their degraded counterpart. In a particular example, a chemiluminescent-labelled oligonucleotide is exposed to chemical conditions which bring about hydrolysis of the chemiluminescent molecule and thus loss of chemiluminescence. If however duplex formation (hybridisation) with a complementary sequence has occurred then chemiluminescence is retained subsequent to attempted hydrolysis due to the protection imparted by the environment of the duplex. The same principles can be applied to fluorescent molecules since it is known that structural modification can alter fluorescent properties as in the cleavage of the carbohydrate residue from 4-methylumbelliferyl-β-D-galactose (Ishikawa and Kato, Scand J Immunol 8 (Suppl. 7)1978).

### SUMMARY OF THE INVENTION

In contrast to the prior art, we have developed systems which are capable of discriminating between the nucleic acids that constitute the substrate of a reaction, and the product molecules that are formed as a result of the action of enzymes acting on the said nucleic acids. In such actions the enzymes will cause a change of state in the substrate.

Preferred embodiments of the invention are designed to be extremely sensitive to apparently minor structural changes in the substrate. For example preferred embodiments are capable of detecting a “nick” in a nucleic acid even where there are no bases missing in the nucleic acid. It will however be appreciated that the generation of or failure to repair such a nick may have major consequences in the replication, recombination and repair etc. of DNA and RNA. Likewise the preferred embodiments provide the ability to detect insertion, deletion, transposition of one or more bases or sequences in DNA or RNA as well as changes in the non-covalent structure thereof.

Accordingly, in one aspect, this invention provides a method for determining the activity of a substance capable of altering the structure of a nucleic acid from a first state to a second state, which comprises the steps of:
(a) providing in a test sample;
   (i) said substance,
   (ii) said nucleic acid; and optionally
   (iii) one or more oligonucleotides complementary, at least in part, to said nucleic acid when in said first or second state wherein;
      either, or both, of said oligonucleotide or nucleic acid have associated therewith a label capable of providing an output signal, and further wherein the stability of said label against degradation is different depending upon whether said nucleic acid is in said first or second state;
(b) exposing said test sample to degradation conditions;
(c) detecting said output signal and thereby determining whether said nucleic acid is, at least predominantly, in said first or second state; and thereby
(d) determining the activity of said substance.

The above method essentially detects a change in state of a nucleic acid caused by substance activity.

Reference herein to the term activity includes reference to increased, decreased or zero activity of said substance.

Embodiments of the invention provide an efficient and reliable means of measuring the activity or inhibition of activity of substances involved in nucleic acid metabolism, and particularly in the repair and replication of genetic material. In preferred embodiments the methodology uses labelled oligonucleotide sequences.

In some embodiments the labelled oligonucleotide sequences differentiate between the first and second state nucleic acid molecules appropriate to these substances (i.e. between the two states of the nucleic acid) by selective binding (hybridisation) to the product (second state) molecule which in turn affects the chemical properties of the luminescent molecule. In other embodiments the labelled oligonucleotide sequences may selectively hybridise to the unmodified (first state) molecule. In yet other embodiments, the labelled oligonucleotide sequences are pre-prepared as a contrived nucleic acid where the labelled oligonucleotide can be thought of as already present in the nucleic acid molecule.

The definition of nucleic acid as used in the present invention includes DNA, RNA, cDNA, gDNA, mRNA, tRNA, multi-stranded DNA, for example double or triple stranded DNA, as well as mixtures of such nucleic acids. The term nucleic acid used herein also encompasses strands of DNA or short sequences or even a collection of unligated individual nucleic acid bases.

The “State” of a nucleic acid and its change from one state to the other refers to characteristics such as for example only whether a strand thereof is either intact or nicked; whether selected bases or sequences thereof have been transposed in one or more strands; whether the duplex has been unwound; whether the duplex has been cleaved; whether the non-covalent structure has changed; whether strands thereof have been integrated; whether strands thereof have been ligated; whether the collection of relevant bases has been assembled into a sequence. For convenience herein, the state of a nucleic acid is not deemed to change again once it has been subjected to substance activity. Thus if a nicked duplex is repaired by a ligase enzyme the duplex is said to have changed from a first (nicked) state to a second (repaired) state. If in a method described herein the two strands of repaired duplex are subsequently separated, they are still regarded as being in the second state.

The term “hybridise” means the formation of a stable duplex or other multiple-stranded molecule between complementary single stranded molecules.

Embodiments of the invention provide simple, rapid and robust assays to measure the activity of substances having an affect on nucleic acid metabolism. Whilst useful in many situations where the assessment of such activity is required, these assays are particularly suitable for the screening of putative anti-bacterial and anti-viral compounds capable of inhibiting the said substance activity.

Where substance activity is assessed, said substance may be one or more of ligase, nuclease, transposase, integrase, primase, helicase, gyrase, polymerase, reverse transcriptase, a topoisomerase or an enediyne.

Enediynes are naturally occurring organic molecules (for e.g. calicheamicin and esperamicin) that behave as restriction endonucleases as they have the ability to cleave duplex nucleic acid and it is this ability to convert a nucleic acid molecule from a first to a second state that enables these molecules to be included within the scope of this invention. Whilst their action is non-catalytic they have a 1:1 reaction with a nucleic acid molecule. The enediyne class of molecules have been described in detail in the following publication (Borders D B & Doyle T W 1995 ‘Enediyne Antibiotics as Anti-Tumour Agents’ (Dekker, New York)) and their ability to mimic the activity of restriction endonucleases is described in the following paper: Biggins et al PNAS 97 13537-13542.

It therefore follows that enediynes are substances falling within the scope of the invention since they are able to convert a nucleic acid molecule from a first to a second state and thus, using the technology described herein, the activity of these molecules can be assayed. Furthermore, using the invention described herein the presence of these molecules, and thus the presence of their activity within a sample, can also be identified. Furthermore, given the ability of these molecules to alter the molecular structure of a nucleic acid from a first to a second state it also follows that, using the invention described herein, it is possible to screen for molecules that regulate the activity of enediynes and so identify molecules or agents which are active pharmacologically as agonists or antagonists thereof.

In a particular embodiment for monitoring the activity of ligase, said nucleic acid is multi-stranded and step (a) involves exposure of a double-stranded nucleic acid to ligase and after exposure to the ligase, the sample is subjected to a raised temperature to cause any unligated nucleic acid, at least partially, to yield single strands.

The use of temperature control selectively to melt or selectively to re-hybridise unligated nucleic acid fragments provides an opportune way of differentiating between ligated and unligated nucleic acid. In one technique, the raised temperature is controlled so that unligated nucleic acid at least partially separates but ligated nucleic acid does not.

As noted above the invention may be used for monitoring a wide range of different enzymes. Thus in another embodiment, for monitoring helicase activity, the nucleic acid is multi-stranded and step (a) involves exposing the nucleic acid to helicase in an environment which allows at least partial unwinding of the nucleic acid.

In a preferred methodology for determining the activity of helicase said oligonucleotide, referred to herein above, is omitted from the test sample and, instead, said nucleic acid is provided with said label.

Where the nucleic acid is multi-stranded and the enzyme is a helicase the nucleic acid is changed between first and second states, by:
(i) separating at least a portion of one of the strands of the nucleic acid from another thereof to provide a single strand portion; and optionally,
(ii) contacting the sample with one of said oligonucleotides, wherein one of said oligonucleotides or said nucleic acid has associated therewith said label, and said oligonucleotide is capable of hybridising to said single strand portion of the nucleic acid.

In yet another embodiment, for monitoring the activity of a polymerase or primase, the nucleic acid is in the form of nucleotides or short fragments thereof and part (a) involves exposure of said nucleic acid to a polymerase in an environment which allows said bases and/or nucleic acid strands to join.

It will be appreciated that the assay methods may be designed to provide one of two different endpoints; in one, the label of the labelled nucleic acid is relatively affected if said nucleic acid has undergone a change in state; in the other, the label of the labelled nucleic acid is relatively unaffected if said nucleic acid has undergone a change in state.

Where said enzyme when active acts to repair at least one of a nick or other discontinuity in an interrupted strand of a multi-stranded nucleic acid, to provide a repaired strand, part (a) may comprise the steps of:
(i) raising the temperature of the sample to a temperature in excess of the temperature required to cause the interrupted strand to separate from the or each remaining strand, (irrespective of whether the interrupted strand has been repaired);
(ii) contacting the sample with said labelled oligonucleotide, said labelled oligonucleotide being capable of hybridising to the repaired strand;
(iii) reducing the temperature of the sample to a temperature below the melting point of a duplex containing the repaired strand, but above the melting point of a duplex containing the non-repaired portions of said interrupted strand, thereby to allow said labelled oligonucleotide to hybridise to said repaired interrupted strand if present; and
(b) thereafter exposing said sample to said degradation conditions and subsequently detecting the activity of the label,
whereby in the said detection step, the presence or amount of relatively unaffected label indicates the presence or amount of activity respectively of said repair enzyme.

In this arrangement, hybridisation of the labelled oligonucleotide to the repaired strand when present, results in a complex in which the label is relatively protected against degradation.

Of course a similar method may be used where, instead of repairing a nick or discontinuity, an enzyme when active generates at least one of a nick or other discontinuity by inter-base cleavage in at least one target strand of a multi-stranded nucleic acid to create an interrupted target strand. In this instance, part
(a) may comprise:—
   (i) raising the temperature of the sample to a temperature in excess of the temperature required to cause the target strand of the nucleic acid to separate from the or each remaining strand (irrespective of whether the enzyme has been active to create an interrupted target strand);
   (ii) contacting the sample with said labelled oligonucleotide, said labelled oligonucleotide being capable of hybridising to said target strand when in uninterrupted form;
   (iii) reducing the temperature of the sample to a temperature below that at which said uninterrupted target strand will hydridise to the labelled oligonucleotide, but above the temperature at which the separated interrupted portions of the target strand can hybridise to said remainder of the nucleic acid, thereby to allow said labelled oligonucleotide to hybridise to said uninterrupted target strand if present, and;
   (b) thereafter exposing said sample to said degradation conditions and subsequently detecting the activity of the label,
   whereby in said detection step, the presence or amount of relatively affected label indicates the presence or amount respectively of said enzyme capable of yielding a cleaved molecule.

In this assay, if uninterrupted strands are present in the sample, the labelled oligonucleotide will hybridise thereto in step (iii) to form a complex in which the label is relatively protected.

In each of the above instances, the sample may be contacted with the labelled oligonucleotide before or after the step of raising the temperature (Step (i)).

In another aspect or embodiment of the invention, said labelled nucleic acid may comprise a complex made up of said nucleic acid and a label, said nucleic acid being capable of being acted upon by a substance whereby, on said substance being active, the nucleic acid changes from said first state to said second state, thereby changing the stability of the label. Such a complex is referred to elsewhere herein as a contrived substrate.

In another embodiment, said nucleic acid is in the form of a collection of free (i.e. unligated) nucleotides, and said enzyme is active to cause or allow selected free nucleotides to be joined to yield a second state in which they form at least one strand of a product nucleic acid, and part (a) involves contacting said sample with a labelled oligonucleotide designed to hybridise with said product nucleic acid.

In another embodiment said substance is a nuclease or an enediyne, said oligonucleotide is omitted from said sample and said nucleic acid, which is multi-stranded and includes a cleavage point is provided with said label and step (a) further includes subjecting said sample to a temperature that causes any cleaved nucleic acid to separate into single strands. Alternatively, this embodiment of the invention may be modified such that said oligonucleotide is not omitted from said sample and thus said nucleic acid is not provided with said label. In this embodiment of the invention said nuclease or enediyne acts on said nucleic acid thus cleaving same so that, when said sample is subject to a temperature that causes any cleaved nucleic acid to separate into single strands said labelled oligonucleotide can bind to a selected one of said strands for the purpose of carrying out the assay.

The detection of the output signal from the label assay may involve the use of one or more of colourimetric, fluorimetric or chemiluminescent means.

The label may conveniently be a fluorescent or chemiluminescent molecule, for example an acridinium salt.

As well as detecting substance activity, the methods disclosed herein may be used for screening for modulatory activity. Thus in another aspect this invention provides a method for screening an agent for modulatory activity in relation to a substance capable of altering the structure of a nucleic acid from a first state to a second state, which comprises the steps of:
(a) providing in a test sample:
   (i) said substance;
   (ii) said nucleic acid;
   (iii) an agent to be tested; and optioinally
   (iv) at least one oligonucleotide complementary, at least in part, to said nucleic acid, when in said first or second state wherein;
   either, or both, of said oligonucleotide and said nucleic acid has associated therewith a label capable of providing an output signal, and further wherein the stability of said label against degradation is different depending on whether said nucleic acid is in said first or second state;
(b) exposing said test sample to degradation conditions;
(c) detecting said output signal and thereby determining whether said nucleic acid is, at least predominantly, in said first or second state; and thereby
(d) determining the activity of said substance and thus the modulatory activity of said agent.

The above method may be used to screen substances for pharmacological activity.

The invention also extends to a nucleic acid for use in detecting the activity of a predetermined substance, said nucleic acid being capable of reactivity with said substance and having an associated label, the location of the label and the configuration of the nucleic acid being selected such that, in use, when said substance is active on said nucleic acid it changes the state of the nucleic acid from a first state to a second state, and wherein the stability of said label against degradation in a subsequent reaction is different according to whether said nucleic acid is in its first or second state.

The invention also extends to a method of detecting whether a nucleic acid in a sample has undergone an event resulting in said nucleic acid changing from a first state to a second state,

As noted previously, the use of selective temperature management provides an important way of detecting whether an enzyme creates or repairs a nick in a substrate nucleic acid.

In another aspect this invention provides a method for detecting in a sample the activity or presence of an enzyme capable of repairing an interrupted nucleic acid strand to form a repaired nucleic acid strand, which comprises the steps of:—
(a) providing in said sample;
   (i) a multi-stranded nucleic acid having an interrupted target strand made up of at least two interrupted portions capable of being ligated by said enzyme when active;
(b) applying the sample to a temperature in excess of the melting temperature of at least one of the interrupted portions of the unrepaired interrupted target strand, but below the melting temperature of the repaired interrupted strand, whereby there is little or no hybridisation of at least one of the unrepaired interrupted portions of the target strand to the complementary strand or strands, and
(c) thereby determining at least one of the activity or presence of said enzyme.

In another aspect this invention provides a method for detecting in a sample the activity or presence of an enzyme capable of generating a nick or other discontinuity in at least one target strand of a multi-stranded nucleic acid to create an interrupted target strand, which comprises the steps of:
(a) providing in said sample;
   (i) a multi-stranded nucleic acid incorporating a site at which a nick or discontinuity may be generated or created;
(b) applying the sample to a temperature in excess of the melting temperature of at least one of the unligated portions of the interrupted target strand (if present), whereby there is little or no hybridisation of said at least one of the unligated portions of the interrupted strand to the complementary strand or strands and;
(c) thereby determining at least one of the activity or presence of said enzyme.

In either of the above determining steps the methodology may include introducing into the sample a labelled oligonucleotide complementary to at least a portion of the said complementary strand of the nucleic acid, thereby to detect the presence or amount of hybridisation between the repaired or uninterrupted strand and said complementary strand. Alternatively said determining step may include introducing into the sample a labelled oligonucleotide complementary to one of the fragments of interrupted strand thereby to detect the presence or amount of hybridisation.

In one arrangement said nucleic acid is selected with regard to the interrupted fragments or the active site such that there are three different melting temperatures as follows:
(i) a melting temperature of a first fragment of the interrupted strand of the substrate;
(ii) a melting temperature of a second fragment of the interrupted strand of the substrate;
(iii) a melting temperature of an uninterrupted strand of the substrate.

It will be appreciated that the melting temperatures of the fragments and their lengths may be controlled in various ways, for example by their relative lengths, or by introducing selected mismatches in the sequences.

### DETAILED DESCRIPTION OF VARIOUS EMBODIMENTS

In one embodiment, an oligonucleotide sequence is labelled with a chemiluminescent molecule that can be rendered non-chemiluminescent by dissociation of one or more bonds but is protected from said dissociation when the labelled oligonucleotide sequence constitutes part of a multi-stranded nucleic acid, for example a duplex. Surprisingly we have found that, under the conditions used to bring about dissociation of the chemiluminescent molecule, a nucleic acid containing an unligated strand is incapable of offering protection against dissociation.

Thus in embodiments described below a labelled chemiluminescent oligonucleotide, is synthesised which is complementary to the sequence of interest, the sequence of interest being the substrate or product of the enzyme or enediyne of interest. A solution of the labelled oligonucleotide is admixed with a solution of the said sequence of interest under conditions conducive to hybridisation. The reaction mixture is then exposed to chemical, enzymatic and/or physical degradation conditions known to bring about dissociation of the chemiluminescent molecule and thus render it non-chemiluminescent. The reaction vessel is then placed in a luminometer and reagents added to bring about the chemiluminescent reaction whilst monitoring any emitted light. Alternatively, if the kinetics of the chemiluminescent reaction are sufficiently slow, the chemiluminescence can be initiated prior to placing the reaction vessel into the luminometer. The presence of the sequence of interest and so the formation of a duplex with the labelled oligonucleotide results in retention of chemiluminescence whereas the absence of the sequence of interest and so the inability to form a duplex with the labelled oligonucleotide results in the loss of chemiluminescence. Consequently, it is possible to determine the relative amounts of the sequence of interest.

One embodiment provides an assay for ligase or nuclease enzymes or enediynes, since the substrate and product molecules differ by being ligated or unligated sequences. Thus, for example, if “nicked” DNA is exposed to a preparation possessing ligase activity the formation of ligated product will be revealed by hybridisation to the chemiluminescence labelled oligonucleotide and retention of chemiluminescence due to protection from, for example, conditions capable of hydrolysing uncomplexed chemiluminescent label.

Preferably, the nucleic acid in a sample is exposed to ligase and after exposure to the ligase, the sample is subjected to a raised temperature to cause nucleic acid in the sample to denature or separate, and subsequently the temperature is reduced to allow the nucleic acid to rehybridise.

It is preferred that the raised temperature used is high enough such that unligated nucleic acid separates but ligated nucleic acid does not. Preferably, the temperature used is adjusted according to the stoichiometry of the hybridisation reaction.

A surprising finding is that in many cases the enzyme or enediyne is capable of functioning even when the nucleic acid to be acted upon possesses a label moiety. Thus a further aspect of the invention defined above involves the use of a pre-formed, labelled enzyme or enediyne ‘substrate’ (referred to as a contrived substrate) which comprises a multi-stranded e.g. a double-stranded oligonucleotide sequence wherein one of the strands possesses a hydrolysable chemiluminescent label as described above.

Optionally, said other strand possesses a ‘nick’. Upon exposure to elevated temperature, for example, the unligated duplex is incapable of protecting the chemiluminescent label from hydrolysis whereas the ligated duplex, formed as the result of prior ligase activity, protects the chemiluminescent label from hydrolysis.

The embodiments described herein disclose ways of assessing the activity of enzymes responsible for the interconversion of ligated and unligated forms of genetic material which are potential targets for the screening of putative pharmacologically active compounds.

Similarly, the same principles can be applied to the assay of those enzymes which catalyse the insertion (integrase) or transposition (transposase) of discrete nucleotide sequences within a given gene sequence. Here use is made of an appropriate labelled oligonucleotide sequence which is capable of hybridising with the product sequence but not the substrate sequence. In this way, not only can the activity of integrase or transposase preparations be assessed but it is possible to determine whether chemical compounds added into the reaction mixture are capable of inhibiting the enzyme activity and may thus have utility as pharmacological agents.

Enzymes of the class exemplified by nuclease, ligase, integrase and transposase all have the common feature of catalysing the covalent modification of genetic material.

There also exist enzymes which catalyse changes in secondary structure of the genetic material, such enzymes being exemplified by helicase. Activity of these enzymes results in the formation of sections of “unwound” nucleic acid. Here, use is made of the fact that the “unwound” product nucleic acid sequence produced as a result of the enzyme activity is accessible to binding by a complementary labelled oligonucleotide sequence in contrast to the substrate duplex nucleic acid sequence.

As above it may be desired to use a pre-formed substrate including double-stranded nucleic acid and already containing the luminescent labelled oligonucleotide sequence and in which the luminescent label is protected from degradation (e.g. hydrolysis) due to its position within the double stranded nucleic acid. The presence of helicase activity then causes the duplex nucleic acid to be unwound hence exposing the luminescent label to hydrolysis. In this case, luminescence intensity is inversely proportional to helicase activity.

In a further preferred embodiment of the invention, the nucleic acid is exposed to helicase in an environment which allows unwinding of strands making up the nucleic acid, and a material is included in the sample which could alter activity of the helicase, and then conditions are provided for the strands to rehybridise in the presence of labelled oligonucleotides complementary to one strand of the above unwound nucleic acid.

In certain situations, as a variation of the situation when labelled oligonucleotide sequence binding is used subsequent to performing the enzymic reaction, it may be appropriate to design the labelled oligonucleotide sequence to bind to the substrate rather than the product of the enzyme reaction.

The inventive principles herein can also be applied to those situations where a nucleic acid product is created from small precursors such as individual bases since the product of the enzyme reaction is capable of hybridisation with a labelled complementary oligonucleotide sequence whereas the reactants are not. Examples of such enzymes are primase, polymerase and reverse transcriptase.

In another embodiment of the invention, nucleic acids/strands are exposed to polymerase in an environment which allows nucleic acids/strands to join, and included in the sample are one or more nucleic acids/strands which may be complementary to the joined nucleic acids/strands and providing conditions for said joined and complementary nucleic acids/strands to hybridise.

Normal enzyme activity gives rise to a nucleic acid capable of hybridisation with a complementary labelled oligonucleotide sequence and the subsequently formed duplex protects the label from degradation. Inhibition of the enzyme results in no duplex being formed and hence no protection of the label from induced degradation. The subsequent measurement of luminescence of a marker such as a chemiluminescent or fluorescent label on the oligonucleotide is therefore a quantitative indicator of the activity or otherwise of the enzyme concerned.

Further, luminescent labels also have the advantage that it is possible to configure “multichannel” assays. There exist in the literature reports of using both wavelength and temporal discrimination to enable mixtures of labels to be quantified simultaneously yet independently (U.S. Pat. No. 5,827,656). This same principle can be used to good effect in the present teachings where, for example, it may be desirable to screen chemical compounds simultaneously for inhibitory activity toward for e.g. ligase and integrase. Based upon the disclosures herein, one skilled in the art will readily appreciate how suitable multichannel assays may be designed and used.

### BRIEF DESCRIPTION OF THE DRAWINGS

FIG. 1 shows a schematic diagram of the action of a ligase enzyme in which parts of a nucleic acid sequence are ligated;

FIG. 2 shows a schematic diagram of the action of a helicase enzyme in which the individual strands of a double-stranded nucleic acid are “unwrapped”;

FIG. 3 is a schematic diagram representing the steps involved in a first embodiment of this invention to assay for ligase activity;

FIG. 4 is a schematic diagram representing the steps involved in a second embodiment of this invention also to assay for ligase activity;

FIG. 5 is a schematic diagram representing the steps involved in a third embodiment of the invention whereby a contrived labelled substrate nucleic acid is used to assay for ligase activity;

FIG. 6 is a schematic diagram representing the steps involved in a fourth embodiment of the invention to assay for activity of enzymes such as DNA helicase;

FIG. 7 is a schematic diagram representing the steps involved in a fifth embodiment of the invention to assay for enzymes such as RNA polymerase active on a multi-stranded DNA template;

FIG. 8 is a schematic diagram representing the steps involved in a sixth embodiment of the invention to assay for activity of enzymes such as reverse transcriptase or primase which act on a single-stranded template;

FIG. 9 is a schematic diagram representing the steps involved in a seventh embodiment of the invention to assay for the activity of enediynes or enzymes, such as nucleases, which are active on multi-stranded DNA;

FIG. 10 is a schematic diagram representing the steps involved in an eighth embodiment of the invention to assay for the activity of enzymes such as integrases which act on oligonucleotides;

FIG. 11 is a schematic diagram representing the steps involved in a ninth embodiment of the invention to assay for the activity of enzymes such as topoisomerases which act on double-stranded nucleic acid molecules;

FIG. 12 shows the results of the experiment of Example 1 where EDTA is used as an inhibitor of the ligase enzyme;

FIG. 13 shows the results of the experiment of Example 2 where di-deoxy thymidine triphosphate (ddTTP) is used as an inhibitor of the reverse transcriptase enzyme;

FIG. 14 shows the results of the experiment of Example 3 where the activity of helicase is measured at 3 enzyme:substrate ratios;

FIG. 15 shows the results of Example 4 where EDTA is used as an inhibitor of the viral DNA dependent RNA polymerase enzyme; and

FIG. 16 shows the results of the experiment of Example 5 where rifampicin is used as an inhibitor of the bacterial DNA dependents RNA polymerase enzyme.

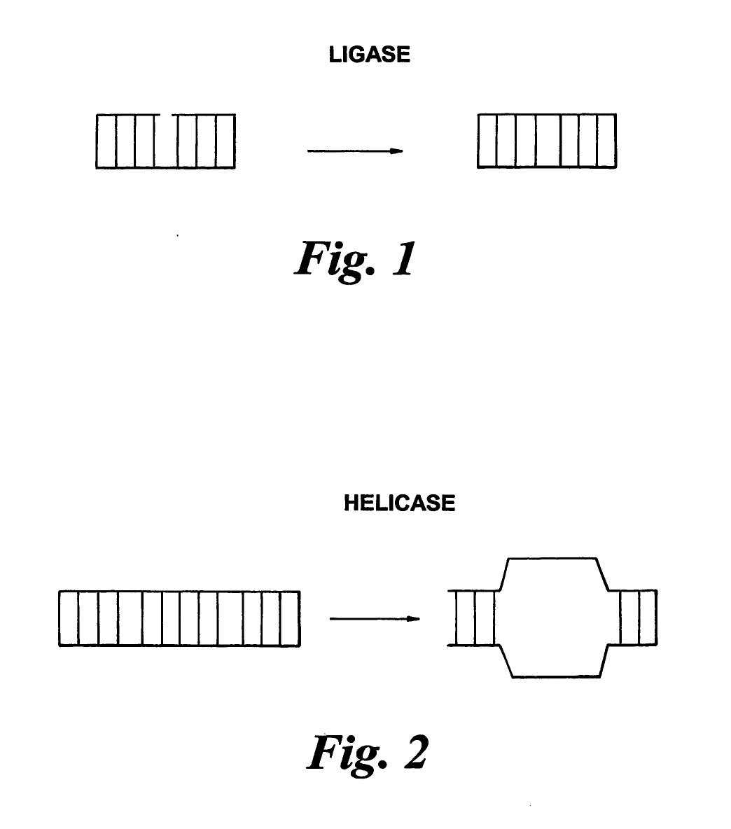

In various embodiments of this invention the change in state of a substrate nucleic acid is detected by causing the formation of a complex made up of one of the strands of the substrate nucleic acid (which may or may not be the strand directly affected by the change in state) and a labelled oligonucleotide which is designed so that its protection against degradation in a subsequent degradation step is different according to whether the substrate nucleic acid is in its first or second state. Following exposure to a degradation step, the label signal is detected in a manner appropriate to the label being used, and from this may be determined the state of the substrate nucleic acid.

For a better understanding of the various techniques we now describe a number of different schemes, with reference to the schematic diagrams in the Figures.

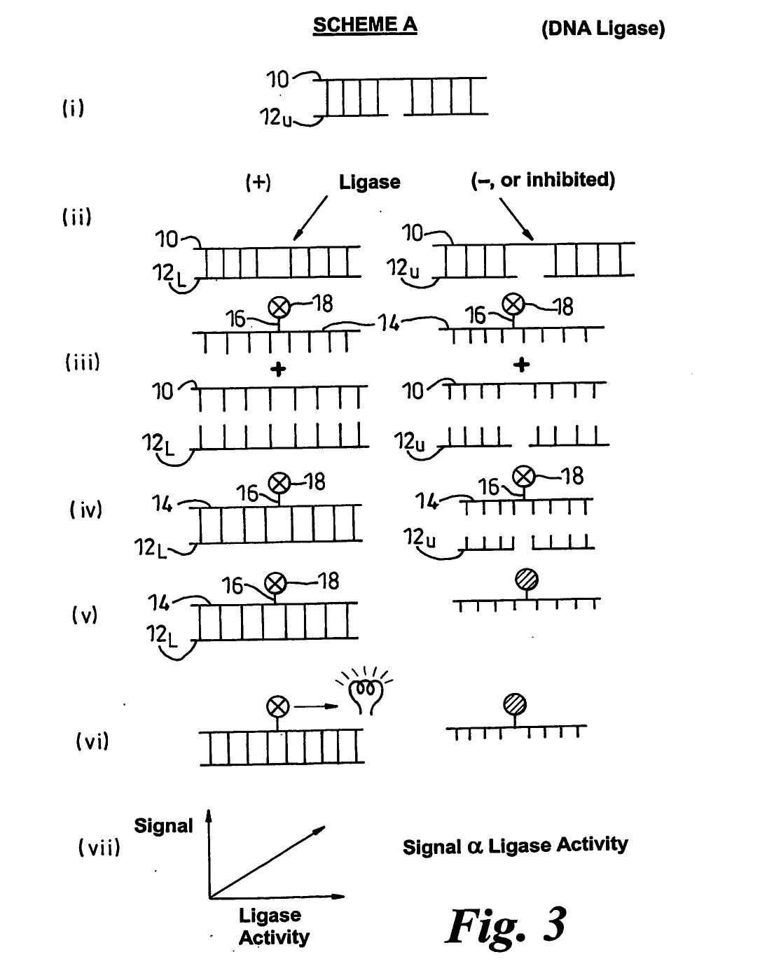

Scheme A (FIG. 3)

A first oligonucleotide duplex is synthesised which comprises a first strand 10 of nucleotides complementary to a second strand 12. When bound in a nucleic acid duplex with the first strand, the second strand can exist either as an intact (ligated) strand 12_{L} or a “nicked” unligated strand 12_{U}. For the purposes of this scheme, which is designed to detect ligase activity, or factors influencing such activity, the nucleic acid duplex is synthesised with the second strand 12_{U} nicked or unligated. The unligated second strand 12_{U} represents at least part of a strand capable of acting as a ligase enzyme substrate which is converted to the ligated strand 12_{L} by the action of the enzyme. In this assay, the two states of the substrate nucleic acid are the one in which the second strand is unligated, and the one in which the strand is ligated (ii).

A third oligonucleotide 14 is synthesised which is identical to the first strand 10 (and thus complementary to the second strand 12), but which further comprises a “linker” moiety 16 to which can be attached a chemiluminescent or fluorescent emitter molecule 18. In certain applications it may not be necessary for the third oligonucleotide to be identical to the first strand; base mismatches may be allowed provided that the third oligonucleotide is capable of hybridising stably to the second strand.

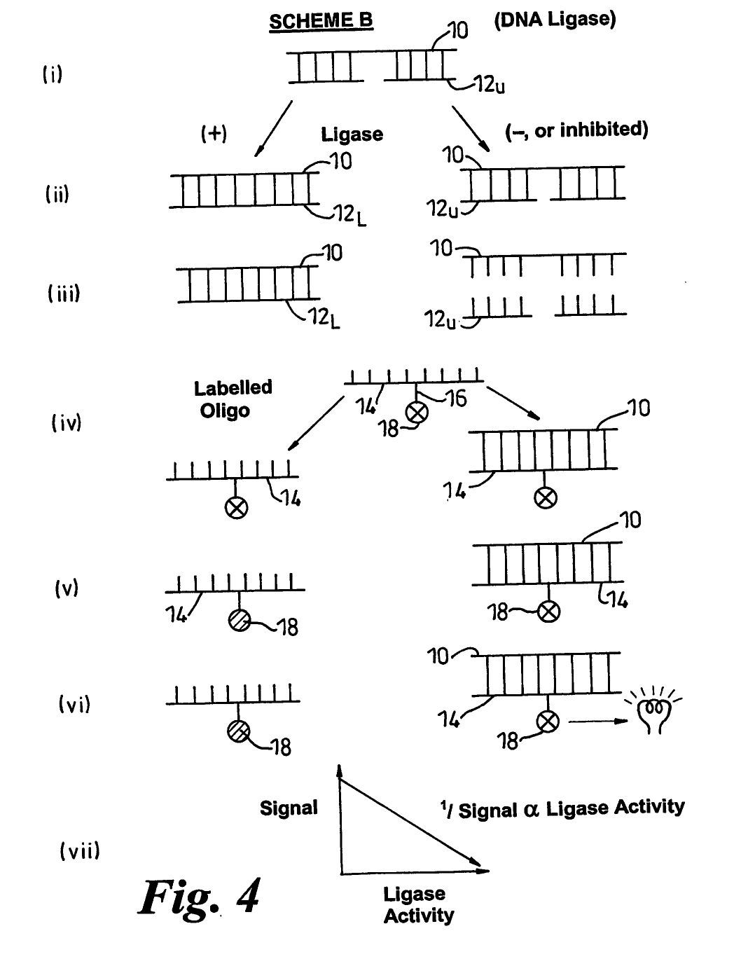

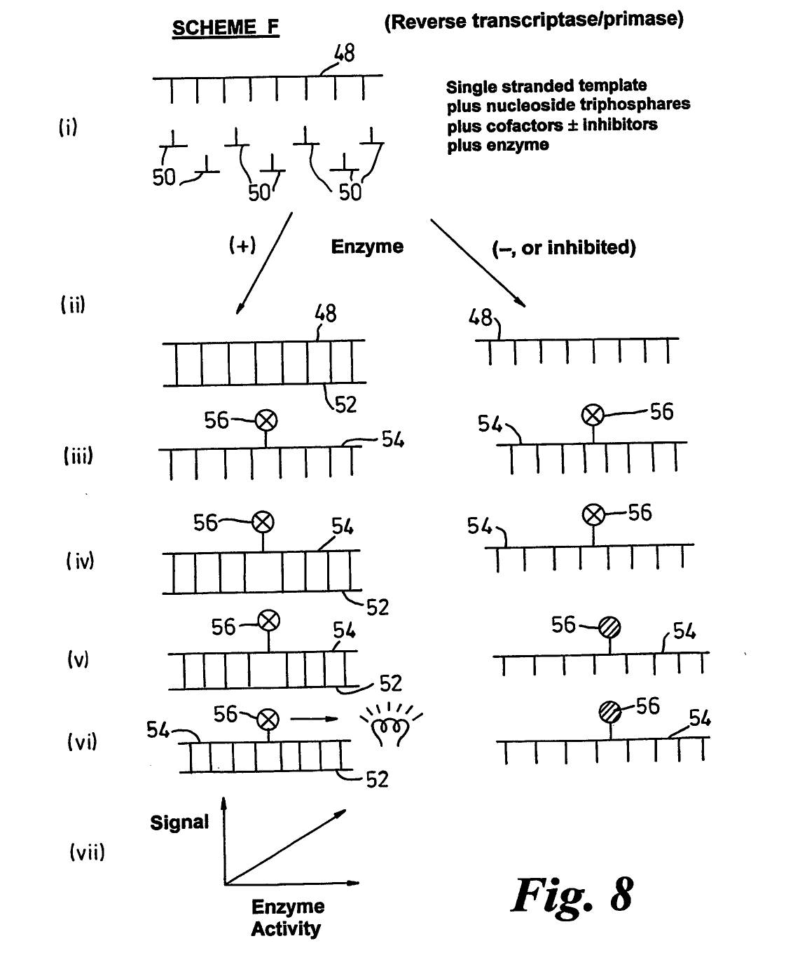

Scheme A comprises the following stages, in which the bracketed roman numerals relate to the steps illustrated in FIG. 3.
(i) A reagent is provided consisting of duplex strands of the first strand 10 hybridised to the second strand 12_{U}, the second strand 12_{U} being “nicked” or unligated.
(ii) The reagent of step (i) is exposed to a ligase enzyme with or without inhibitors and co-factors. (The left hand side of the Figure shows the condition where there is enzyme activity and the right hand side shows the condition where there is no such activity; this also applies in the remainder of FIGS. 3 to 8).
(iii) A labelled oligonucleotide 14 is introduced into the sample, and the temperature of the sample is raised to cause the first and second strands to separate.
(iv) The temperature is reduced to a temperature below the hybridisation temperature of the ligated (intact) strands 12_{L} but above the temperature of the unligated fragments of the second strand 12_{U}, so that some of the second ligated strands 12_{L} will hybridise to the labelled oligonucleotide 14 instead of to the first strand 10. However, as the temperature is above the hybridisation temperature of the unligated short target strands, the fragments of the unligated 12_{U} will not hybridise to the labelled oligonucleotide 14.
(v) The sample is then subjected to conditions which degrade the label 18, e.g. by hydrolysis or dissociation of the label (hereinafter referred to generally as degradation conditions). The nature of the labelled oligonucleotide is such that, if the labelled oligonucleotide has hybridised to an intact strand 12_{L}, the output signal from the label 18 will be substantially unaffected. On the other hand, if the labelled oligonucleotide has not hybridised (or has only partially hybridised), it will not be protected against the degradation conditions and so the light output signal will be affected (it may be non-existent or it may be in an altered form).
(vi) The chemiluminescent reaction is initiated and the light output is measured or fluorescence is measured depending on the nature of the label.
(vii) The light output signal is proportional to ligase activity.
   Scheme B—FIG. 4

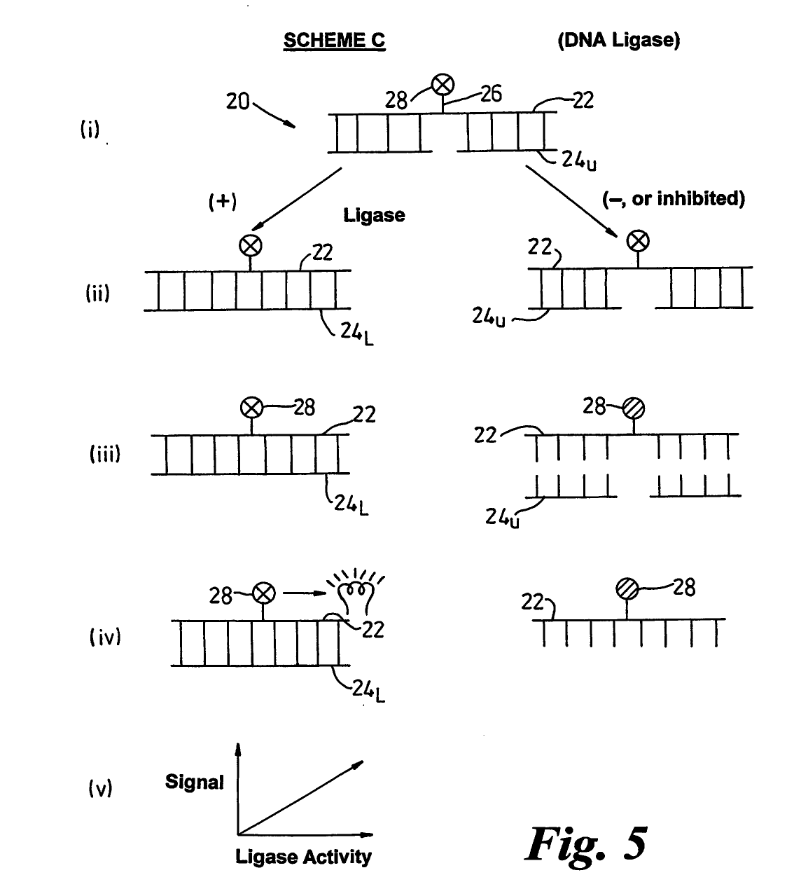

This scheme is similar to Scheme A in that it uses a first strand 10 and a second strand 12 which may be in ligated form (12_{L}) or unligated form (12_{U}), and a labelled oligonucleotide is used. However in this example, the labelled oligonucleotide 14 is designed to hybridise with the first strand 10 rather than the second strand.
(i) Substrate duplex strands made up of the first strand 10 hybridised to the second strand 12_{U} in unligated form are provided in the sample.
(ii) The sample is exposed to ligase with or without inhibitors, co-factors etc.
(iii) The temperature of the sample is raised to a temperature high enough to cause unligated second strands 12_{U} to separate from the first strand, but not high enough to cause ligated second strands 12_{L} to separate.
(iv) The sample is exposed to a labelled oligonucleotide 14 complementary to the first strand and hybridisation is allowed to occur to any of the unhybridised first strands 10.
(v) The sample is subjected to degradation conditions such that the chemiluminescent or fluorescent activity of any unhybridised labelled oligonucleotide 14 is affected but that the activity of any labelled oligonucleotide 14 hybridised to complementary strand (in this instance the first strand 10) is substantially unaffected.
(vi) The chemiluminescent reaction is initiated and the light output is measured or fluorescence is measured depending on the nature of the label.
(vii) The light output signal is inversely proportional to ligase activity.
   Scheme C—(FIG. 5)

In this scheme a contrived substrate nucleic acid duplex 20 is engineered.
(i) The substrate nucleic acid duplex 20 is made up of a first strand 22 hybridised to a nicked or unligated second strand 24_{U}. A linker moiety 26 connects a label 28 to the first strand 22. The contrived substrate nucleic acid duplex 20 is designed with the label 28 positioned relative to the nick in the unligated strand 24_{U} such that the label is relatively unprotected against degradation conditions whilst the second strand is unligated but is relatively protected against such conditions if the second strand is ligated by enzyme activity. Whilst in the schematic representation the label is shown directly opposite the nick, the relative locations of the label and the nick in the unligated strand may be varied and indeed the nick may be several bases away from the location of the label on the opposite strand. Suitable location of the nick relative to the label and to the ends of the contrived substrate may be determined empirically, based on the disclosures of U.S. Pat. Nos. 5,283,174 and 5,399,491.

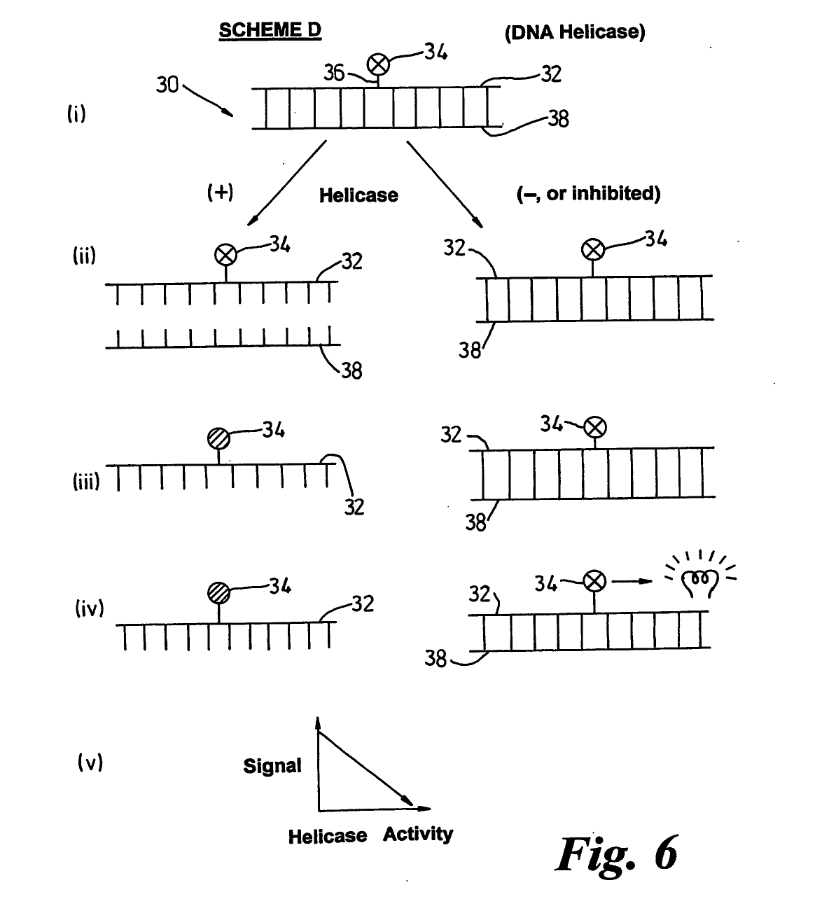

(ii) The contrived substrate 20 is exposed to ligase with or without inhibitors, co-factors etc. In the presence of ligase activity, the unligated second strand 24_{U} is repaired to provide a ligated strand 24_{L}. In the absence of enzyme activity the second strand 24_{U} is unrepaired.
(iii) The sample is then raised to a temperature sufficiently high to cause unligated second strands 24_{U} to separate from the first strand 22 but not high enough to cause ligated second strands 24_{L} to separate from the first strand 22. The sample is then subjected to degradation conditions to degrade the activity of the label 28 if the first strand is not protected by the ligated second strand 24_{L}.
(iv) The chemiluminescent reaction is initiated and the light output is measured or fluorescence is measured depending on the nature of the label.
(v) The light signal output is proportional to ligase activity.
   Scheme D—(FIG. 6)

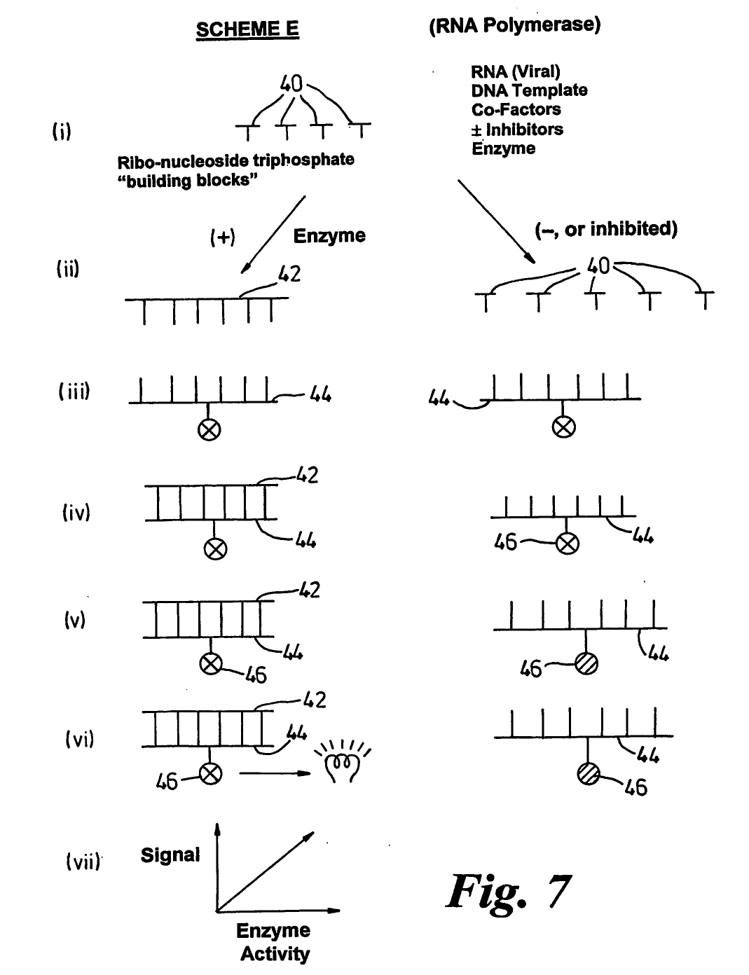

This scheme is intended for monitoring for activity of an enzyme such as DNA helicase which causes separation of two strands.
(i) A contrived duplex strand 30 is provided with a first strand 32 having a label 34 attached by means of a linker moiety. The first strand 32 is hybridised to a second strand 38.
(ii) The contrived substrate 30 is exposed to helicase with or without inhibitors, co-factors etc. In the presence of active helicase the first and second strands 32 and 38 are separated by enzyme activity to change the state of the duplex but, in the absence of such activity, the state of the contrived substrate 30 is unaltered.
(iii) The sample is exposed to degradation conditions to degrade the activity of the chemiluminescent or fluorescent label 34. If the first strand 32 has become separated from the second strand 38 then the activity of the label 34 will be compromised, but if the enzyme is not active the label 34 will be relatively protected.
(iv) The sample is subjected to conditions to cause loss of chemiluminescent or fluorescent activity if unprotected.
(v) The light output signal is inversely proportional to helicase activity.
   Scheme E—(FIG. 7)

This Scheme is useful for monitoring activity of an enzyme such as RNA polymerase or other enzymes which assemble the ribo-nucleoside triphosphate “building blocks” 40 into a nucleic acid sequence 42.
(i) A suitable duplex DNA or RNA template (not shown) is provided in a sample together with ribo-nucleoside triphosphates 40, the enzyme being tested and any required co-factors or inhibitors.
(ii) If the enzyme is uninhibited it assembles a single stranded ribo-nucleotide product 42; otherwise the ribo-nucleoside triphosphates 40 remain separate.
(iii) A labelled oligonucleotide 44 complementary to the product of enzyme activity of (ii) is introduced into the sample.
(iv) The labelled oligonucleotide 44 hybridises to the assembled product 42 if present.
(v) The sample is subjected to degradation conditions to cause loss of chemiluminescent or fluorescent activity. Where the labelled oligonucleotide 44 hybridises to the product 42 the stability of the label 46 is relatively unaffected as compared to where the labelled oligonucleotide has no assembled strand to which to hybridise.
(vi) The chemiluminescent reaction is initiated and the light output is measured or fluorescence is measured depending on the nature of the label.
(vii) The light output signal is proportional to enzyme activity.
   Scheme F

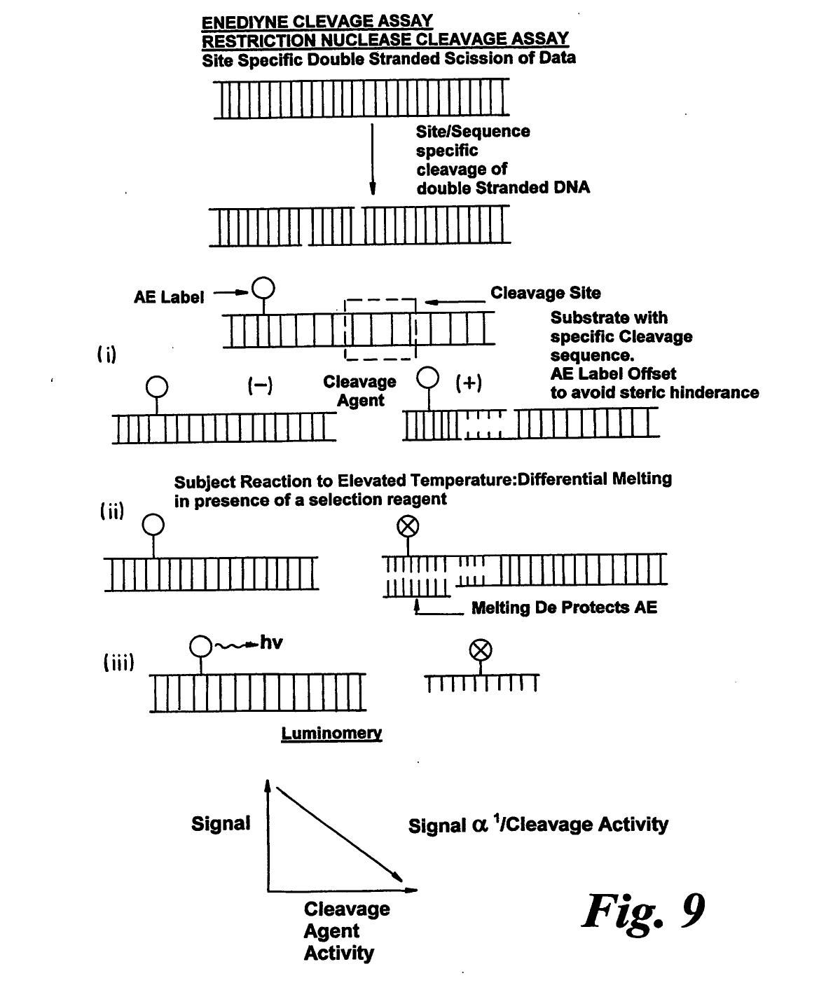

This scheme is designed for monitoring activity etc. of enzymes such as reverse transcriptase or primase.
(i) A sample is made up comprising a single-stranded template 48 together with nucleoside triphosphates 50, the enzyme being tested, and one or more co-factors or inhibitors if required.
(ii) Where active, the enzyme generates a complementary target strand 52 on the template 48; where inactive no complementary strand is generated.
(iii) A labelled oligonucleotide 54 complementary to the enzyme-synthesised target strand 52 is introduced into the sample.
(iv) The temperature is cycled to cause the template 48 and the enzyme-synthesised target strand 52 to separate and then lowered to allow hybridisation; if the target strand 52 is present, some of these will hybridise to the labelled oligonucleotide 54.
(v) The sample is subjected to degradation conditions to cause loss of chemiluminescence or fluorescent activity such that unhybridised labelled oligonucleotide loses activity.
(vi) The chemiluminescent reaction is initiated and the light output is measured or fluorescence is measured depending on the nature of the label.
(vii) The light output signal from the label 50 is proportional to enzyme activity.
   Scheme G (FIG. 9)

(a) DNA duplex which comprises a site specific cleavage point is first synthesised. A chemiluminescent label (AE label) is then attached to one of the strands of the duplex at a site sufficiently remote from the said cleavage point so that the label will not interfere with the activity of a nuclease enzyme. Most ideally, the said label is positioned so as to avoid any steric hindrance between itself and the nuclease enzyme. If a cleavage agent or nuclease enzyme is not present the labelled duplex will remain substantially intact (left-hand side of FIG. 9). Alternatively, if a cleavage agent or nuclease enzyme is present it will act upon the site specific cleavage point, cleaving the DNA. Thus, when the DNA is subsequently exposed to a suitably selected melt temperature the cleaved strand separates away from its complementary strand leaving the chemiluminescent label exposed. Thereafter, when exposed to hydrolysing conditions the chemiluminescent label is destroyed. In contrast, where the enzyme is absent, or inactive, cleavage of the duplex does not occur and thus the chemiluminescent label can shelter from the effects of hydrolysis within the duplex coil and so retain its functionality. In this way, the output of the chemiluminescent signal is inversely proportional to cleavage activity. Thus as cleavage increases, due to the increased presence or activity of the nuclease enzyme, more and more chemiluminescent label is destroyed and so the chemiluminescent signal declines.

Scheme G also illustrates the steps involved in the enediyne cleavage assay. As above, in the presence of an enediyne the labelled duplex is cleaved and subsequently, upon exposure to melt conditions, if cleavage has taken place a cleaved strand separates away from its complementary strand leaving the chemiluminescent label exposed. Thereafter, when exposed to hydrolysing conditions the chemiluminescent label is destroyed. It therefore follows that this assay method is equally effective at assaying for the activity or presence of an enediyne.

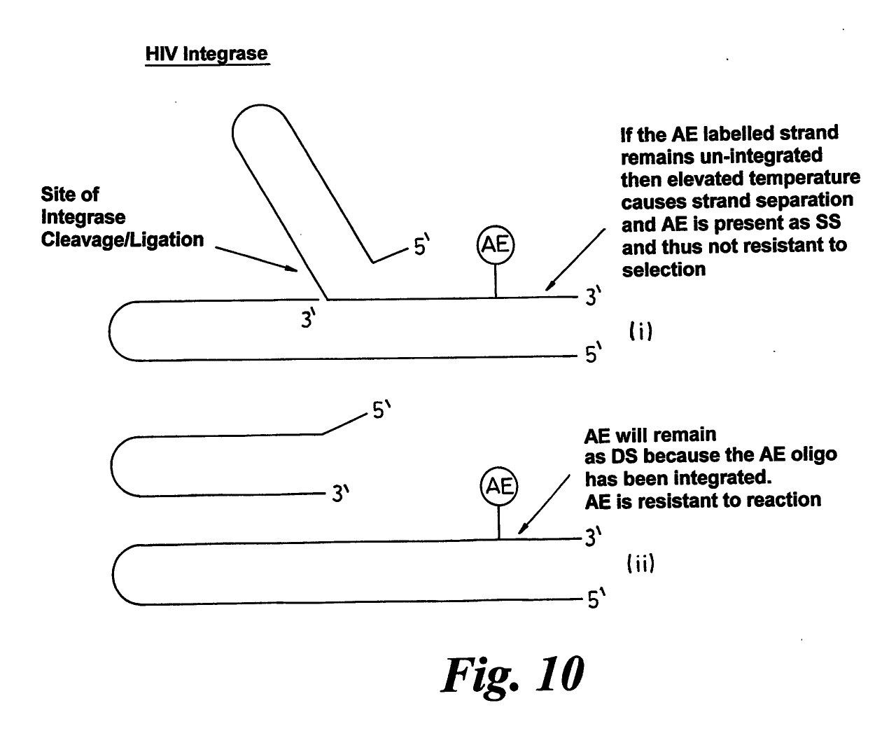

In a modification of the Scheme shown in FIG. 10 the chemiluminescent label may not be attached to the duplex but instead provided on a separate oligonucleotide which is complementary, at least in part, to a portion of the duplex whereby in the presence of the enzyme or the enediyne the duplex is cleaved and the oligonucleotide can bind to its complementary portion of the duplex. Thus, in this variation of Scheme G, the binding of the labelled oligonucleotide to a fragment of the duplex is indicative of the presence of the enzyme or the enediyne and proportional to the activity thereof.

Scheme H (FIG. 10)

This scheme is designed for monitoring the activity of intergrase enzymes.

The substrate for this enzyme consists of two oligonucleotides. They contain inter and intra complementary sequences which are able to hybridise to produce the secondary structure depicted. Intergrase cleaves and ligates this structure such that a chemiluminescent (AE) labelled strand is incorporated into the larger of the two oligonucleotides. Upon exposure to elevated temperature the unincorporated, or smaller, oligonucleotide melts off. The larger oligonucleotide is then exposed to hydrolysing conditions and the chemiluminescent label, ligated into the long strand by the intergrase, can take shelter in the coil of the double stranded nucleic acid and so resist degradation.

In this scheme the signal of the chemiluminescent label is directly proportional to the activity of the intergrase enzyme. Thus, the more active the enzyme, the more chemiluminescent label is incorporated into duplex and so the more it can be protected from the degradative effects of hydrolysis.

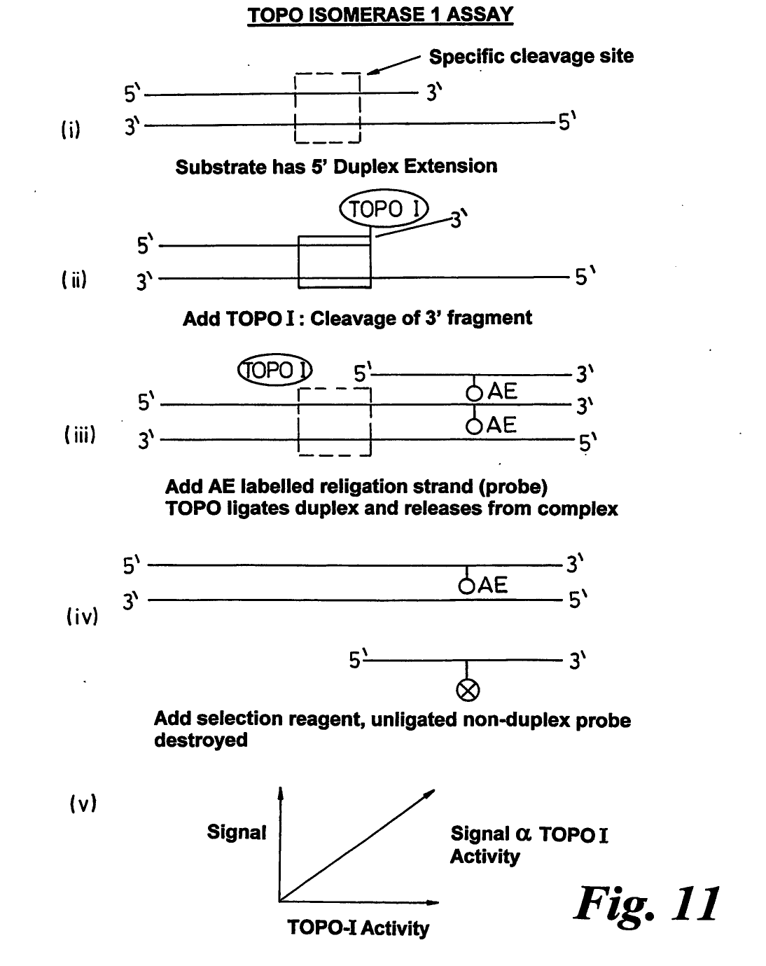

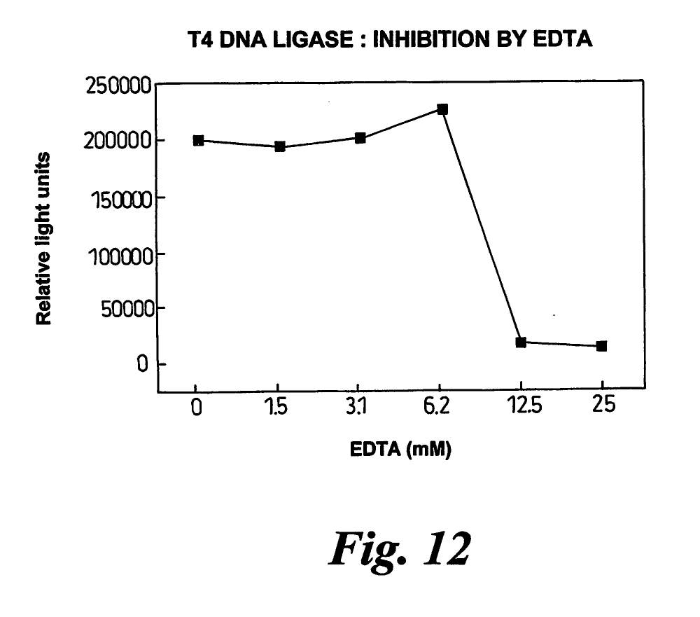

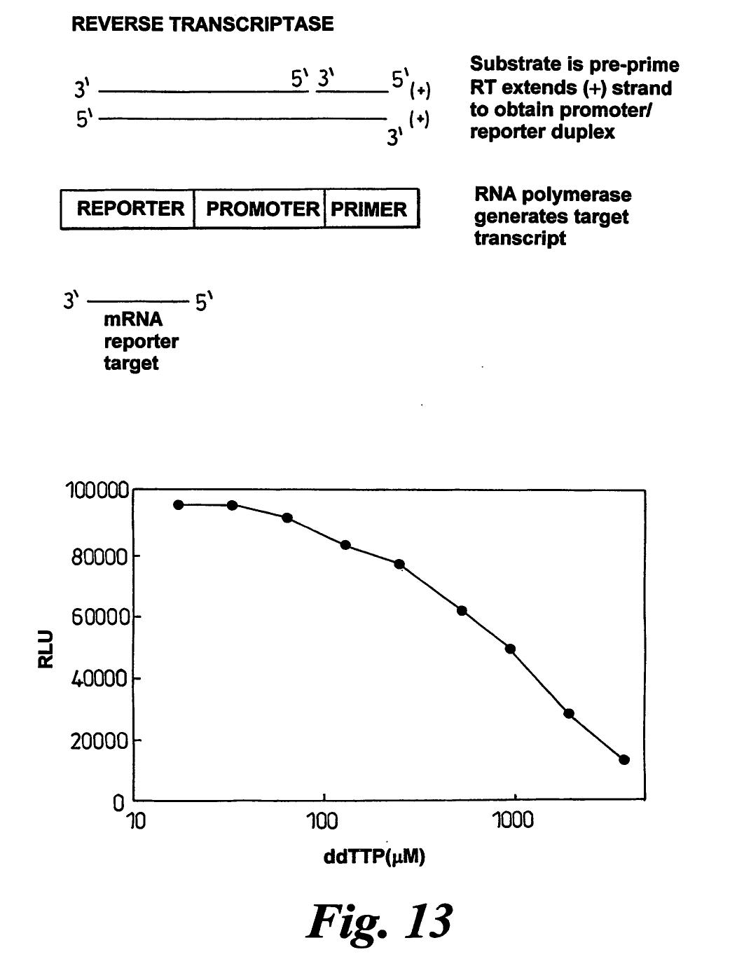

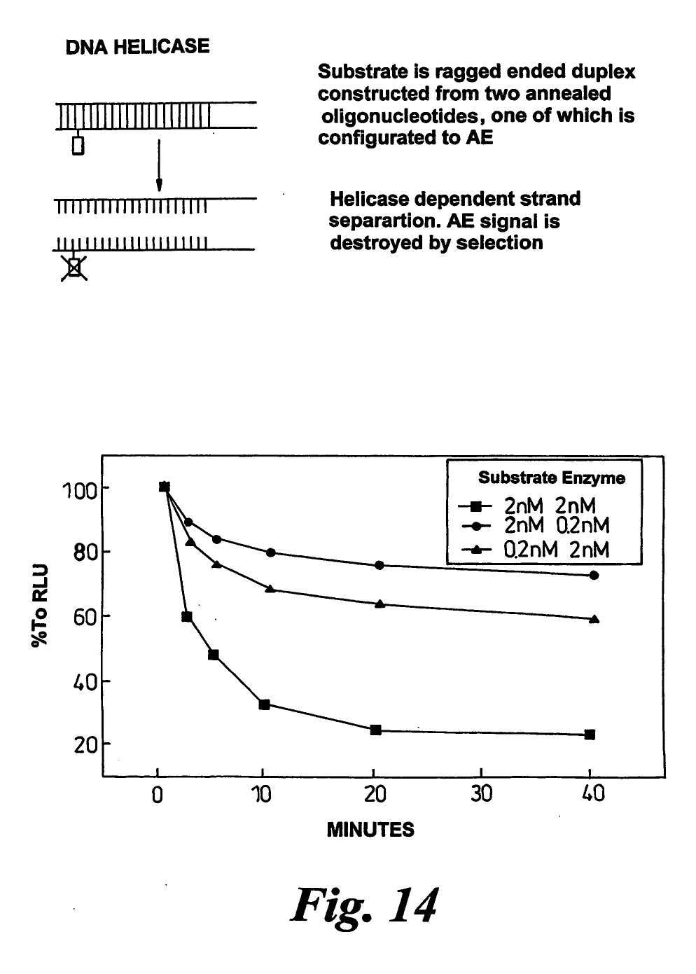

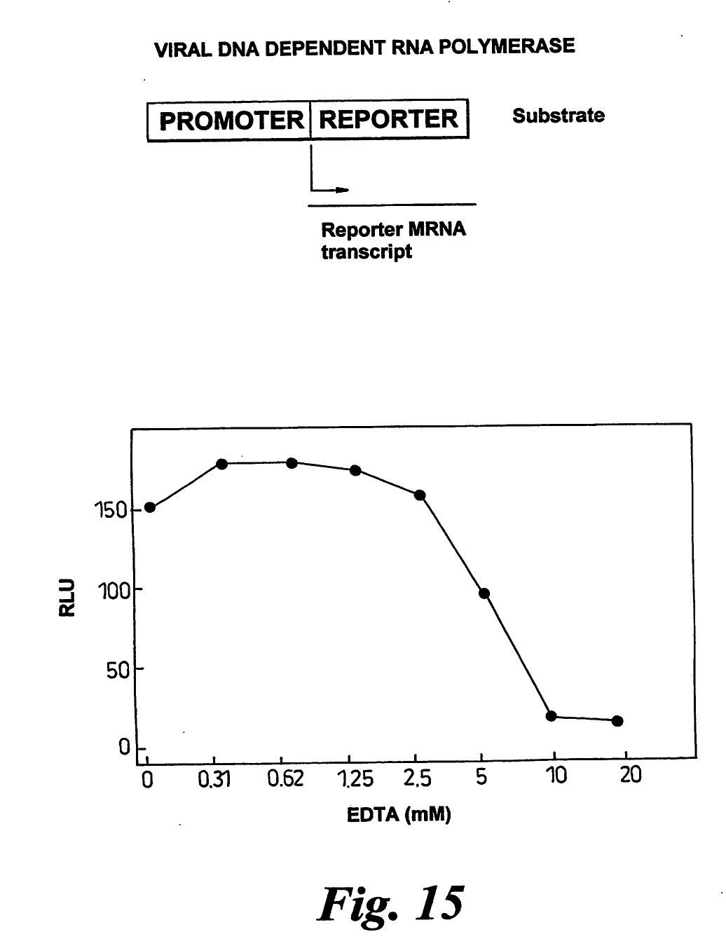

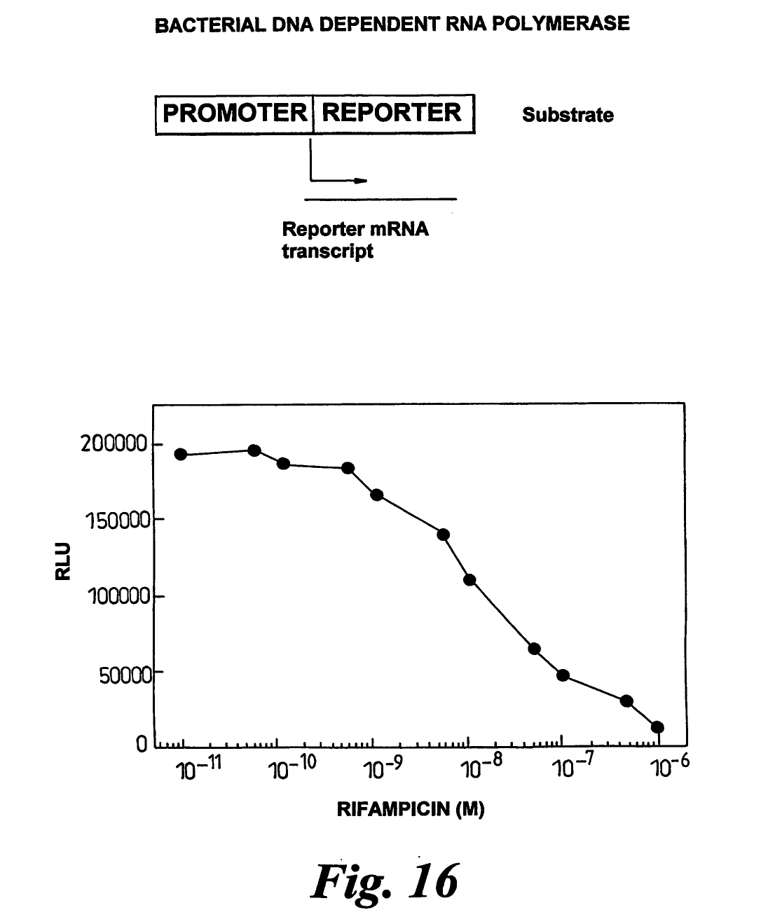

Scheme I (FIG. 11)

This scheme is designed for monitoring the activity of topoisomerase enzymes.

A duplex nucleic acid with a 5′ duplex extension is first manufactured. One of the strands of this duplex further includes a specific cleavage site for the enzyme topoisomerase. If topoisomerase is present or active, then it acts upon the duplex, at the cleavage site, to produce a duplex with an extended 5′ extension.

A chemiluminescent labelled oligonucleotide, complementary to said extended 5′ extension is then added to the assay. The topoisomerase then ligates this oligonucleotide thus producing a chemiluminescent labelled duplex.

Upon exposure to degradation conditions, by way of hydrolysis, said chemiluminescent label is protected from hydrolysis in the coil of the duplex. In contrast, any unligated oligonucleotide is destroyed.

In this assay the signal intensity of the chemiluminescent label is directly proportional to the activity of the topoisomerase enzyme. Thus, the amount of signal increases as the enzyme acts to incorporate the oligonucleotide label into the extended duplex.

### DETAILED DESCRIPTION OF THE INVENTION

Indirect Ligase Activity Assay Based on Scheme ‘A’

Here a first oligonucleotide strand is synthesised which comprises a sequence of nucleotides complementary to a second (target) strand present in nicked or unligated form. The unligated second strand represents at least part of a strand capable of acting as a ligase enzyme substrate which is converted to a repaired or ligated strand by the action of the enzyme. The strand is “nicked” preferably at a position where the ratio of the relative lengths of the two components of the unligated strand does not exceed four. The possible range of positions of the nick is constrained by the overall length of the nicked strand. The third oligonucleotide strand has a nucleotide strand identical to the said first oligonucleotide strand but which further comprises a “linker” moiety to which can be attached a chemiluminescent or fluorescent emitter molecule. The synthesis of such labelled oligonucleotides is well-established. Preferably the first and third oligonucleotide strands comprise nucleotide strands of between 10 and 60 bases, more preferably between 20 and 40 bases. Preferably the emitter molecule is a chemiluminescent molecule, more preferably the emitter molecule is a chemiluminescent acridinium salt.

A suitable ligase substrate is prepared by admixture of said first and second strands such that a nicked duplex is produced similar to that in step (i) of Scheme A. In practice the second strand comprises two shorter strands one of which is phosphorylated at its free 5′-end by a suitable method for example by using T4 polynucleotide kinase. Preferably 10-100 nmol of each strand is hybridised in suitable buffer, preferably lithium succinate 1-100 mmol/l, 0.1-1 ml for preferably 0.5-2 hours at 60° C. A suitable amount of this substrate is then admixed with the desired amount of enzyme and the reaction allowed to proceed for an appropriate period of time under the usual conditions.

The labelled third oligonucleotide strand is dissolved in a buffer medium which is compatible with the labelled strand in terms of allowing it to hybridise to the second oligonucleotide strand and in terms of maintaining the stability of the reagents during the hybridisation reaction. The formulation of such buffers is established in this field. Typically the buffer ions consist of organic and/or inorganic salts preferably at concentrations in the range 1 to 100 mmol/l and the solutions may contain other solutes such as surfactants and/or preservatives and possess pH values preferably of seven or less. The amount of labelled oligonucleotide used depends on the sensitivity of detection of the label and the sensitivity of detection of target strand required in the assay. It is known that, typically, chemiluminescence emission can be more sensitively detected than conventional fluorescence emission and that therefore fluorescent probes may be inappropriate where very high sensitivity of detection is required. The amount of labelled oligonucleotide used for an individual determination may typically lie in the range 10⁻¹⁸ to 10⁻⁹ mol, more preferably 10⁻¹⁵ to 10⁻¹² mol. This may be contained in a volume of buffer in the range 1 microlitre to 1 millilitre, though this could be less than 1 microlitre in certain situations.

The solution of labelled probe is admixed with the analytical sample in a suitable reaction vessel such as a discrete test tube, or part of an array of reaction vessels such as a 96, 384 or 1536 well microtitre plate. Alternatively it is known that many analysis procedures make use of solid-phase systems involving the use of immobilised microarrays and it will be appreciated that the means described herein can be extended to such systems in parallel to the manner in which conventional labelled probe assays have been used.

The hybridisation reaction is allowed to proceed at a temperature typically in the range 4-80° C., more preferably in the range 30-60° C. for a period of time typically in the range 1 minute to 240 minutes, more typically 5 minutes to 30 minutes.

Following the first incubation there a degradation stage in which there is added to the reaction mixture a degradation reagent capable of causing one or more bonds in the label moiety to dissociate in such a manner that where the label is part of an intact duplex it is protected from the said dissociative process. The dissociative processes generally also require the use of elevated temperatures. The degradation reagent may be a buffer solution with a pH greater than 7 which is capable of bringing about hydrolysis of the label moiety. The invention is not limited to the use of hydrolysis and extends to other ways of selectively inhibiting the ability of the emitter label to produce light depending on whether the emitter label is part of an intact duplex or not. Examples of other ways of performing such selective dissociation reactions are disclosed in the literature (Ishikawa and Kato). In this technique, the intensity of chemiluminescence emission is proportional to the ratio of ligated to unligated nucleic acid.

In the above assay to determine ligase activity with a DNA enzyme substrate, the hybridisation reaction is preceded by a reaction step in which the enzyme, if present, acts to cause changes in the structure of a nucleic acid. In the case of ligase, this involves repairing “nicks” in the nucleic acid. The nucleic acid is then heated to denature or separate the hybridised strands and subsequently cooled to allow the strands to rehybridise. Where it is desired to determine whether or not a compound or mixture of compounds is capable of inhibiting or activating the enzyme activity, the said enzyme is exposed to the said compound or mixture of compounds and its activity, or lack thereof, as assayed is compared with the assayed enzyme activity of enzyme not so exposed. In a similar manner the activity of any chemical or physical system causing the conversion of “substrate” to product can be determined as can the activity of inhibitors or activators thereof.

Direct Ligase Activity Assay—Based on Scheme C

A “contrived” enzyme substrate is produced comprising a double-stranded oligonucleotide strand having between 20 and 60 base pairs, and one of the strands possessing at least one “nick” such that the nicked strands are unligated. Furthermore, one of the strands of the nicked strand possesses a linker and hydrolysable chemiluminescent label as described above. The substrate is used in an assay for ligase enzyme activity in which the substrate and enzyme are admixed under conditions appropriate for the particular ligase enzyme being used, and which ensure that the double-stranded substrate does not dissociate into single strands during the enzyme reaction.

Subsequent to the exposure of the substrate to the enzyme, the reaction mixture is exposed to an elevated temperature typically in the range 35 to 75° C., more preferably in the range 45 to 65° C. in order to hydrolyse any unprotected chemiluminescent label. Such hydrolysis is also facilitated where necessary by prior addition of an appropriate buffer solution to raise the pH of the reaction mixture preferably within the range 7 to 9.

Following the selective hydrolysis step, the reaction mixture is placed in a luminometer where the chemiluminescence emission is initiated and measured. The method of initiation of the chemiluminescent reaction is dependent on the particular chemiluminescent label being used, such methods being known to those skilled in the art. In one example where the label is a chemiluminescent acridinium salt, the initiation is typically effected by the addition of hydrogen peroxide and alkali. A wide range of suitable instruments for chemiluminescence detection is commercially available.

Whilst the procedures described above relate to monitoring ligase activity, they may be used for any enzyme which facilitates the interconversion of ligated and unligated nucleic acids. These procedures will start with, or be preceded by, a method in which the enzyme being tested is mixed with the nucleic acid substrate under conditions and in the presence of any co-factors necessary for the reaction to proceed. Also at this point, or earlier, there may be added a substance to be investigated as to its possible effect on the activity of the said enzyme.

The reaction conditions compatible with the activity of a given enzyme are well established in the literature and can be applied to the teachings herein. Moreover the general procedures which represent the preferred modes for bringing about the interactions between enzymes and inhibitors are well-known. Accordingly, the techniques disclosed herein may be adapted to allow for the study of any chemical or physical variable affecting the activity of the enzymes described herein.

Ultimately, the intensity of chemiluminescence is proportional (either directly or indirectly depending on the methodology) to the ratio of the concentration of ligated to unligated strand and as such is an indication or measure of the activity, inactivity or inhibition of activity of the enzyme present in the system.

The methods described can be applied as a means of determining the activity of a range of enzymes which are responsible for the modification of nucleic acid and which involve ligation and/or cleavage as part of their overall function. In this situation, the temperature at which the hydrolysis procedure is carried out needs appropriate selection since it must also permit unligated duplex to melt and yet allow ligated duplex to remain intact and thus facilitate hybridisation protection. Appropriate temperatures will be different for different strands and an empirical approach is required to optimise this temperature for a given strand.

Similar experimental protocols may be used for the assay of the activity of helicase enzymes or inhibitors thereof except that in these cases the labelled oligonucleotide strand is designed such that it is capable of binding to “unwound” genetic material that constitutes the product of the respective enzyme activity but incapable of binding to substrate as represented by a nucleic acid duplex. Lack of enzyme activity as occurs upon enzyme inhibition by a chemical compound or mixture thereof results in the absence of accessible target for hybridisation of the labelled oligonucleotide strand.

Further, as set out in Scheme D, a helicase assay may utilise a “contrived substrate” in which one of the strands of the substrate duplex is itself labelled such that the properties of the label are different when the duplex has been “unwound” by the enzyme. The contrived substrate duplex may be labelled with e.g. an acridinium ester whose rate of hydrolysis is increased when that part of the nucleic acid strand to which it is linked is separated from its complementary strand by the action of helicase. As described above, the physical/chemical conditions are then altered to selectively hydrolyse the acridinium salt present in the product of the helicase reaction, whilst leaving substantially unaffected that which is present in the form of unreacted substrate. In this case the intensity of chemiluminescence is inversely proportional to enzyme activity.

Similar experimental protocols may be used for the assay of the activity of integrase and transposase enzymes or inhibitors thereof. Here labelled oligonucleotides may be used that are capable of hybridising to the product nucleic acid strand (i.e. that following enzyme activity) but not the unmodified substrate nucleic acid strand, or vice versa.

It will be appreciated that if the substrate or product to be bound to the labelled oligonucleotide strand exists as a duplex then it may be necessary to bring about dissociation of the said duplex before hybridisation with the oligonucleotide probe can take place. Various ways of bringing about such dissociation are well-established in the art.

The following examples are illustrative of the principles, without limitation as to the application, of the teachings embodied herein.

### EXAMPLE 1

1. DNA Ligase Assay Using Hybridisation Protection of a Chemiluminescent Acridinium Ester Labelled Oligonucleotide Strand.

Three oligonucleotides were prepared using established methods. The strands of these were as follows:

| | |
|---|---|
| (i) | 5′-GGC CTC TTC GCT ATT ACG CCA GCT-3′ |
| (ii) | 3′-CCG GAG AAG CGA-5′ |
| (iii) | 3′-TAA TGC GGT CGA-5′ |

Also prepared by published methods was a chemiluminescent derivative of (i) as follows (* represents the position of the chemiluminescent label)

| | |
|---|---|
| (iv) | 5′-GGC CTC TTC GCT*ATT ACG CCA GCT-3′ |

The free 5′-end of (ii) was phosphorylated by established methods. The phosphorylation ensures that the strands are nicked. Stock duplex was formed by hybridising the phosphorylated (ii) with equimolar amounts of (i) and (iii) for one hour at 60° C. in lithium succinate buffer. Investigations of ligase activity were performed using mixtures of the duplex (6 pmol) and T4 DNA ligase (80 units) admixed with putative inhibitors if required.

The reaction product was analysed for ligated product as follows:

Samples of the ligase product reaction mixture were diluted 1000-fold in tris buffer (0.01 mol/l, pH 8.3) for analysis by hybridisation protection assay. 100 ul of the dilutions were added to labelled probe (iv) (50 fmol) diluted in reaction buffer (125 mmol/l lithium hydroxide, 95 mmol/l succinic acid, 1.5 mmol/l EGTA, 1.5 mmol/l EDTA, 8.5% lithium lauryl sulphate, pH 5.2) in 500 ul microcentrifuge tubes. The tubes were incubated at 95° C. for 5 minutes followed by an incubation at 60° C. for 30 minutes. The tubes were cooled to 4° C. and 100 ul of the contents of each tube transferred to corresponding 12×75 mm polystyrene test tubes. Hydrolysis reagent (190 mmol/l sodium borate, 5% Triton X-100, pH 7.6)(300 ul) was then added and the tubes incubated at 60° C. for 10 minutes. The tubes were placed in an ice bath for one minute and then placed in a luminometer (Stratec Biomedical Systems, Pforzheim, Germany) programmed to sequentially inject 200 ul each of Detection Reagents 1 and 11 (Gen Probe Inc., San Diego, USA) with a read time of 5 seconds.

FIG. 9 shows the effect on the enzyme of a known ligase inhibitor (ethylene diamine tetra-acetic acid, EDTA).

2. DNA Ligase Assay Using Hybridisation Protection of a Chemiluminescent Acridinium Ester Labelled Duplex Substrate.

Oligonucleotides (ii), (iii) and (iv) from Example 1 were hybridised in the same way as previously used for strands (i), (ii) and (iii). The stock labelled duplex was then used directly in the ligase assay.

Hydrolysis reagent was added as before and chemiluminescence measurements carried out as described above.

### EXAMPLE 2

Reverse Transcriptase (RT): Inhibition of by Di-Deoxy Thymidine Triphosphate (ddTTP).

Assay template was a pre-primed 81 nt DNA (non-sense) oligonucleotide consisting of sequential primer, T7 viral DNA dependent RNA polymerase promoter and reporter sequences. RT dependent extension of a short pre-hybridised sense strand primer yields double stranded promoter/reporter and enables RT regulated T7 RNA polymerase generation of report mRNA transcript. Template was incubated in buffer containing rTNPs (2 mM), dTNPs (0.1 mM), avian myeloblastosis virus RT, 17 RNA polymerase and serial dilutions of ddTTP. Reporter mRNA product was then measured by HPA (Hybridisation Protection Assay). Briefly, oligonucleotides complementary to the substrate strand, or its complementary counterpart, where hybridised to the corresponding strand of DNA after exposure to a melt temperature.

Hydrolysis reagent was added as before and chemiluminescence measurements carried out as described above.

### EXAMPLE 3

DNA Helicase: Time Course of Strand Separation at Three Enzyme: Substrate Ratios.

AE labelled double stranded substrate was incubated in the presence of enzyme. Unseparated substrate confers hybridisation protection to AE and thus signal intensity is inversely proportional to enzyme activity.

### EXAMPLE 4

T7 DNA Dependent RNA Polymerase Generation of mRNA: Inhibition Dose Response Using EDTA.

Template was PCR generated linearised DNA containing the 17 RNA polymerase promoter and coding for a 295 nt mRNA transcript including reporter target sequence. Template plus enzyme were incubated in serial dilutions of EDTA as model inhibitor. Reporter mRNA product was then measured by hybridisation protection assay, HPA. Briefly, labelled oligonucleotides complementary to the newly formed strand were hibridised to same. Hydrolysis reagent was added as before and chemiluminescence measurements carried out as described above.

### EXAMPLE 5

E coli RNA Polymerase: Inhibition by Rifampicin.

Stock template was constructed from a 64 nt synthetic oligonucleotide coding sequentially (3′-5′ non-sense) for consensus sequence RNA polymerase promoter and reporter mRNA transcript. A short sense strand primer was annealed at the 3′ terminus and the complete duplex extended using Klenow DNA polymerase. Template was incubated in assay buffer using E coli RNA polymerase holoenzyme with serial dilutions of inhibitor in DMSO. Reporter mRNA product was then measured by hybridisation protection assay HPA. Briefly, labelled oligonucleotides were hybridised to the newly formed strand. Hydrolysis reagent was added as before and chemiluminescence measurements carried out as described above.

1. A method for determining the activity of a microbial or viral enzyme capable of altering the structure of a nucleic acid from a first state to a second state comprising the steps of:
(a) providing in a test sample;
(i) said enzyme selected from the group consisting of: a ligase, helicase, polymerase, reverse transcriptase, primase, nuclease, integrase, topoisomerase, transposase and gyrase:
(ii) said nucleic acid; and, optionally,
(iii) one or more oligonucleotides complementary, at least in part, to said nucleic acid when in said first or second state;
wherein either, or both, of said oligonucleotide or said nucleic acid comprises a label capable of providing an output signal, and further wherein the stability of said label against degradation is different depending upon whether said nucleic acid is in said first or second state;
(b) exposing said test sample to degradation conditions;
(c) detecting said output signal and thereby determining whether said nucleic acid is, at least predominantly, in said first or second state; and
(d) determining the activity of said enzyme.

2-3. (canceled)

4. A method according to claim 1 wherein said nucleic acid is DNA or RNA.

5. A method according to claim 1 wherein said nucleic acid is either single stranded or multi-stranded.

6. A method according to claim 1 or 4 wherein said enzyme is a ligase, said nucleic acid in its first state is multi-stranded, and step (a) further comprises subjecting said sample to a temperature that causes said multi-stranded nucleic acid to separate into single strands and so enables any ligated nucleic acid strand, or its complementary strand, to hybridise with said oligonucleotide.

7. A method according to claim 1 or 4 wherein said enzyme is a ligase, said oligonucleotide is omitted from said test sample and said nucleic acid, which is multi-stranded, comprises said label, and step (a) further comprises subjecting said sample to a temperature that causes any unligated nucleic acid, at least partially, to separate into single strands.

8. A method according to claim 6 wherein said temperature is selected so that unligated nucleic acid separates but ligated nucleic acid does not separate.

9. A method according to claim 6, 7 or 8 wherein said nucleic acid comprises an interrupted strand made up of at least two unligated portions capable of being ligated by said enzyme.

10. A method according to claim 1 or 4 wherein said enzyme is a helicase, said nucleic acid in its first state is multi-stranded, and step (a) further comprises subjecting said sample to an environment which allows, at least partial, unwinding of said nucleic acid.

11. A method according to claim 10 wherein said oligonucleotide is omitted from said test sample and said nucleic acid comprises said label.

12. A method according to claim 1 wherein said enzyme is a polymerase, said nucleic acid is in the form of oligonucleotides and/or nucleotides, and step (a) further comprises subjecting said sample to an environment which allows said oligonucleotides and/or said nucleotides to join to form a strand whereby said complementary oligonucleotide can bind thereto.

13. A method according to claim 1 wherein said enzyme is a reverse transcriptase or primase, said nucleic acid is in the form of nucleoside triphosphates, and step (a) further includes comprises subjecting said test sample to an environment which allows said nucleoside triphosphates to join to form a strand whereby said complementary oligonucleotide can bind thereto.

14. A method according to claim 12 or 13 wherein said sample further comprises a nucleic acid template.

15. A method according to claim 14 wherein said template is single stranded.

16. A method according to claim 1 wherein said enzyme is a nuclease, said oligonucleotide is omitted from said sample and said nucleic acid, which is multi-stranded and comprises a site specific cleavage point, comprises said label, and step (a) further comprises subjecting said sample to a temperature that causes any cleaved nucleic acid to separate into single strands.

17. A method according to claim 1 wherein said enzyme is a nuclease, said nucleic acid is multi-stranded and comprises a site specific cleavage points and step (a) further comprises subjecting said sample to a temperature that causes any cleaved nucleic acid to separate into single strands whereby said complementary oligonucleotide can bind to at least a selected one of said strands.

18-19. (canceled)

20. A method according to claim 16 or 17 wherein said temperature is selected so that said cleaved nucleic acid separates but uncleaved nucleic acid does not separate.

21. A method according to claim 1 or 4 wherein said enzyme is an integrase, said oligonucleotide is omitted from said sample and said nucleic acid comprises at least two oligonucleotides containing inter and intra complementary sequences, and further wherein one of said sequences comprises said labels and step (a) further comprises subjecting said sample to a temperature that causes any unincorporated oligonucleotide to separate or melt away.

22. A method according to claim 7, 16 or 21 wherein said label is located remotely from the site at which said enzyme is active whereby said label cannot interfere with the activity of said enzyme.

23. A method according to claim 1 or 4 wherein said enzyme is a topoisomerase, said nucleic acid in its first state comprises a duplex with a 5′ or 3′ extensions and said oligonucleotide is a religation strand, that is a strand that is complementary to the 5′ or 3′ extension produced by the action of said enzyme.

24. A method according to any one of the preceding claims wherein detecting said output signal comprises the use of one or more of colourimetric, fluorimetric or chemiluminescent means.

25. A method according to any one of the preceding claims wherein said label is a fluorescent or chemiluminescent molecule.

26. A method according to claim 25 wherein said label is an acridinium salt.

27. A method according to any one of the preceding claims wherein the activity of more than one enzyme is determined and said method comprises the steps of:
(a) providing in a test sample:
(i) a plurality of enzymes;
(ii) nucleic acid for said enzymes; and, optionally,
(iii) one or more oligonucleotides complementary, at least in part, to said nucleic acid(s) when in said first or second state;
wherein either, or both, of said oligonucleotide or said nucleic acid comprises a plurality of labels, each capable of providing an output signal which signal is stable against the effects of degradation depending upon whether said nucleic acid is in said first or second state, and further wherein the output signal of each label differs whereby the activity of each enzyme can be distinguished;
(b) exposing said test sample to degradation conditions;
(c) detecting said output signals of each label and thereby determining whether said nucleic acid(s) is, at least predominantly, in said first or second states; and
(d) determining the activity of each of said enzymes.

28-32. (canceled)

33. A method for screening an agent for modulatory activity in relation to a microbial or viral enzyme capable of altering the structure of a nucleic acid from a first state to a second state comprising the steps of:
(a) providing in a test sample:
(i) said enzyme selected from the group consisting of: a ligase, helicase, polymerase, reverse transcriptase, primase nuclease, integrase, topoisomerase, transposase and gyrase;
(ii) said nucleic acid;
(iii) an agent to be tested; and, optionally,
(iv) at least one oligonucleotide complementary, at least in part, to said nucleic acid when in said first or second state;
wherein either, or both, of said oligonucleotide and or said nucleic acid comprises a label capable of providing an output signal, and further wherein the stability of said label against degradation is different depending on whether said nucleic acid is in said first or second state;
(b) exposing said test sample to degradation conditions;
(c) detecting said output signal and thereby determining whether said nucleic acid is, at least predominantly, in said first or second state; and
(d) determining the activity of said enzyme and thus the modulatory activity of said agent.

34-37. (canceled)

38. A substrate nucleic acid for use in determining the activity of a predetermined microbial or viral enzyme selected from the group consisting of a ligase, helicase, polymerase, reverse transcriptase, primase, nuclease, integrase, topoisomerase, transposase and gyrase comprising a complex of a nucleic acid and a label, said nucleic acid being capable of being acted upon by said enzyme whereby, on said enzyme being active, the said substrate nucleic acid changes from a first state to a second state thereby affecting the stability of said label against degradation.

39-44. (canceled)
